SEQUENCE LISTING (72 sequences)

SEQ ID NO: 1 - DNA, 62 bp, Artificial Sequence
  synthetic construct
ggcgggtttgggtgcggggtttgggtcgggggtttgggtggcaatgctcgggaaggctactc

SEQ ID NO: 2 - DNA, 62 bp, Artificial Sequence
  synthetic construct
gcggggtttgggtcgggggtttgggtggcgggtttgggtcggatcccgtgagtcgatggttt

SEQ ID NO: 3 - DNA, 62 bp, Artificial Sequence
  synthetic construct
cgggggtttgggtggcgggtttgggtgcggggtttgggtgcgcgcaccgcagtttggtcaat

SEQ ID NO: 4 - DNA, 60 bp, Artificial Sequence
  synthetic construct
gcggggtttgggtcggggtttgggtgcgggtttgggtcggcacgcggcagtcgagttaat

SEQ ID NO: 5 - DNA, 57 bp, Artificial Sequence
  synthetic construct
gcggggtttgggtcgggtttgggtcgggtttgggtcggagcccggtctcatcgttgt

SEQ ID NO: 6 - DNA, 56 bp, Artificial Sequence
  synthetic construct
gcggggtttgggtgggtttgggtgggtttgggtcggtccgacgcaacaatagggca

SEQ ID NO: 7 - DNA, 16 bp, Artificial Sequence
  synthetic construct
gccacccaaacccccg

SEQ ID NO: 8 - DNA, 20 bp, Artificial Sequence
  synthetic construct
gagtagccttcccgagcatt

SEQ ID NO: 9 - DNA, 20 bp, Artificial Sequence
  synthetic construct
aaaccatcgactcacgggat

SEQ ID NO: 10 - DNA, 20 bp, Artificial Sequence
  synthetic construct
attgaccaaactgcggtgcg

SEQ ID NO: 11 - DNA, 20 bp, Artificial Sequence
  synthetic construct
attaactcgactgccgcgtg

SEQ ID NO: 12 - DNA, 20 bp, Artificial Sequence
  synthetic construct
aacaacgatgagaccgggct

SEQ ID NO: 13 - DNA, 20 bp, Artificial Sequence
  synthetic construct
tgccctattgttgcgtcgga

SEQ ID NO: 14 - DNA, 20 bp, Artificial Sequence
  synthetic construct
tcttctagttgtcgagcagg

SEQ ID NO: 15 - DNA, 20 bp, Artificial Sequence
  synthetic construct
taatctaattctggtcgcgg

SEQ ID NO: 16 - DNA, 20 bp, Artificial Sequence
  synthetic construct
tgtgataatttcgacgaggc

SEQ ID NO: 17 - DNA, 20 bp, Artificial Sequence
  synthetic construct
gtgattaagtctgcttcggc

SEQ ID NO: 18 - DNA, 40 bp, Artificial Sequence
  synthetic construct
tttgggtttgccccttttccaatgctcgggaaggctacct

SEQ ID NO: 19 - DNA, 40 bp, Artificial Sequence
  synthetic construct
ttggttggttggttggttggatcccgtgagtcgatggttt

SEQ ID NO: 20 - DNA, 40 bp, Artificial Sequence
  synthetic construct
tgggtttggggtttgggtttcgcaccgcagtttggtcaat

SEQ ID NO: 21 - DNA, 40 bp, Artificial Sequence
  synthetic construct
gtgtgtgtgttgtgtgtgtgcacgcggcagtcgagttaat

SEQ ID NO: 22 - DNA, 40 bp, Artificial Sequence
  synthetic construct
gtgttgttgttgtggtggtgagcccggtctcatcgttgtt

SEQ ID NO: 23 - DNA, 40 bp, Artificial Sequence
  synthetic construct
ggttggttggtttgggtttgtccgacgcaacaatagggca

SEQ ID NO: 24 - DNA, 40 bp, Artificial Sequence
  synthetic construct
gtttgggtttttggttggttcctgctcgacggctagaaga

SEQ ID NO: 25 - DNA, 40 bp, Artificial Sequence
  synthetic construct
tgtgtgtgtgtgggtttgggccgcgaccagaattagatta

SEQ ID NO: 26 - DNA, 40 bp, Artificial Sequence
  synthetic construct
tgtggtggtggtgtgtgtgtgccrcgtcgaaattatcaca

SEQ ID NO: 27 - DNA, 40 bp, Artificial Sequence
  synthetic construct
tttttggggggtgttgttgtgccgaagcagacttaatcac

SEQ ID NO: 28 - DNA, 30 bp, Artificial Sequence
  synthetic construct
ggaaaaggggcaaacccaaaccaaccaacc

SEQ ID NO: 29 - DNA, 30 bp, Artificial Sequence
  synthetic construct
ccaaccaaccaaccaaccaaaaacccaaac

SEQ ID NO: 30 - DNA, 30 bp, Artificial Sequence
  synthetic construct
aaacccaaaccccaaacccacacacacaca

SEQ ID NO: 31 - DNA, 30 bp, Artificial Sequence
  synthetic construct
cacacacacaacacacacaccaccaccaca

SEQ ID NO: 32 - DNA, 30 bp, Artificial Sequence
  synthetic construct
caccaccacaacaacaacaccccccaaaaa

SEQ ID NO: 33 - DNA, 76 bp, Artificial Sequence
  synthetic construct
ggtagccctcactcggcgccaagnnnnactggcgagnnnncttcgcaagnnnnagcggcttggctagccccgaccg

SEQ ID NO: 34 - DNA, 76 bp, Artificial Sequence
  synthetic construct
ggtagccctcactcggcgccaagcccgactggcgagcgcgcttcgcaagggggagcggcttggctagccccgaccg

SEQ ID NO: 35 - DNA, 20 bp, Artificial Sequence
  synthetic construct
atcccgtgagtcgatggttt

SEQ ID NO: 36 - DNA, 20 bp, Artificial Sequence
  synthetic construct
cgcaccgcagtttggtcaat

SEQ ID NO: 37 - DNA, 20 bp, Artificial Sequence
  synthetic construct
cacgcggcagtcgagttaat

SEQ ID NO: 38 - DNA, 20 bp, Artificial Sequence
  synthetic construct
agcccggtctcatcgttgtt

SEQ ID NO: 39 - DNA, 20 bp, Artificial Sequence
  synthetic construct
tccgacgcaacaatagggca

SEQ ID NO: 40 - DNA, 20 bp, Artificial Sequence
  synthetic construct
cctgctcgacaactagaaga

SEQ ID NO: 41 - DNA, 20 bp, Artificial Sequence
  synthetic construct
ccgcgaccagaattagatta

SEQ ID NO: 42 - DNA, 20 bp, Artificial Sequence
  synthetic construct
gcctcgtcgaaattatcaca

SEQ ID NO: 43 - DNA, 20 bp, Artificial Sequence
  synthetic construct
gccgaagcagacttaatcac

SEQ ID NO: 44 - DNA, 20 bp, Artificial Sequence
  synthetic construct
acaggttcagaatcctcgac

SEQ ID NO: 45 - DNA, 20 bp, Artificial Sequence
  synthetic construct
gcacgtcccagattcgttag

SEQ ID NO: 46 - DNA, 20 bp, Artificial Sequence
  synthetic construct
acacgcacggaatagtatcc

SEQ ID NO: 47 - DNA, 20 bp, Artificial Sequence
  synthetic construct
atatgctcggaacgtgtcgc

SEQ ID NO: 48 - DNA, 20 bp, Artificial Sequence
  synthetic construct
ccatcgcacgaatgtagtac

SEQ ID NO: 49 - DNA, 20 bp, Artificial Sequence
  synthetic construct
ctcatagacgaagcgaggac

SEQ ID NO: 50 - DNA, 20 bp, Artificial Sequence
  synthetic construct
tgcacacacgaagacgagac

SEQ ID NO: 51 - DNA, 20 bp, Artificial Sequence
  synthetic construct
gcggcatacgttcgtcaaat

SEQ ID NO: 52 - DNA, 20 bp, Artificial Sequence
  synthetic construct
ccaggcgacgttccgaatat

SEQ ID NO: 53 - DNA, 36 bp, Artificial Sequence
  synthetic construct
gccagtcgtgcttggcatcccgtgagtcgatggttt

SEQ ID NO: 54 - DNA, 36 bp, Artificial Sequence
  synthetic construct
gccagtcgtccttggccgcaccgcagtttggtcaat

SEQ ID NO: 55 - DNA, 36 bp, Artificial Sequence
  synthetic construct
gccagtcgatcttggccacgcggcagtcgagttaat

SEQ ID NO: 56 - DNA, 36 bp, Artificial Sequence
  synthetic construct
gccagtctaacttggcagcccggtctcatcgttgtt

SEQ ID NO: 57 - DNA, 36 bp, Artificial Sequence
  synthetic construct
gccagtgtagcttggctccgacgcaacaatagggca

SEQ ID NO: 58 - DNA, 36 bp, Artificial Sequence
  synthetic construct
gcgaaggaccctcgcccctgctcgacaactagaaga

SEQ ID NO: 59 - DNA, 36 bp, Artificial Sequence
  synthetic construct
gcgaaggtaactcgccccgcgaccagaattagatta

SEQ ID NO: 60 - DNA, 36 bp, Artificial Sequence
  synthetic construct
gcgaaggcatctcgccgcctcgtcgaaattatcaca

SEQ ID NO: 61 - DNA, 36 bp, Artificial Sequence
  synthetic construct
gcgaaggggtctcgccgccgaagcagacttaatcac

SEQ ID NO: 62 - DNA, 36 bp, Artificial Sequence
  synthetic construct
cgcaaggtgtctcgccacaggttcagaatcctcgac

SEQ ID NO: 63 - DNA, 36 bp, Artificial Sequence
  synthetic construct
gccgctgttccttgcggcacgtcccagattcgttag

SEQ ID NO: 64 - DNA, 36 bp, Artificial Sequence
  synthetic construct
gccgctgcaacttgcgacacgcacggaatagtatcc

SEQ ID NO: 65 - DNA, 36 bp, Artificial Sequence
  synthetic construct
gccgctctagcttgcgatatgctcggaacgtgtcgc

SEQ ID NO: 66 - DNA, 36 bp, Artificial Sequence
  synthetic construct
gccgctgggtcttgcgccatcgcacgaatgtagtac

SEQ ID NO: 67 - DNA, 36 bp, Artificial Sequence
  synthetic construct
gccgctgacccttgcgctcatagacgaagcgaggac

SEQ ID NO: 68 - DNA, 36 bp, Artificial Sequence
  synthetic construct
gccagtcgggcttggctgcacacacgaagacgagac

SEQ ID NO: 69 - DNA, 36 bp, Artificial Sequence
  synthetic construct
gcgaagcgcgctcgccgcggcatacgttcgtcaaat

SEQ ID NO: 70 - DNA, 36 bp, Artificial Sequence
  synthetic construct
gccgctcccccatgcgccaggcgacgttccgaatat

SEQ ID NO: 71 - DNA, 17 bp, Artificial Sequence
  synthetic construct
ggtagccctcactcggc

SEQ ID NO: 72 - DNA, 17 bp, Artificial Sequence
  synthetic construct
cggtcggggctagccaa

(19) United States
(12) Patent Application Publication
(10) Pub. No.: US 2006/0269920 A1
(43) Pub. Date: Nov. 30, 2006

(54) Method for obtaining structural information about an encoded molecule

(75) Inventors: Per-Ola Freskgard, Norrkoping (SE); Soren Neve, Lyngby (DK)

Correspondence Address: BROWDY AND NEIMARK, P.L.L.C.;624 NINTH STREET, NW, SUITE 300, WASHINGTON, DC, 20001-5303, US

(73) Assignee: Nuevolution A/S, Copenhagen O (DK)

(21) Appl. No.: 10/545,795

(22) PCT Filed: Feb. 23, 2004
(86) PCT No.: PCT/DK04/00116

§ 371 Date: Jul. 13, 2006

Related U.S. Application Data
(60) Provisional application No. 60/448,480, filed on Feb. 21, 2003.

(30) Foreign Application Priority Data
Feb. 21, 2003 (DK) PA 2003 00269

(51) Int. Cl. C12Q 1/68; C40B 30/06; C40B 40/08

(52) U.S. Cl. 435/6

(57) ABSTRACT

Disclosed is a method for obtaining structural information about an encoded molecule, wherein the encoded molecule has been produced by a process comprising reacting a plurality of chemical entities, said chemical entities being coded for by codons on a nucleic acid template. The method comprises the steps of providing an array comprising a plurality of single stranded nucleic acid probes immobilized in discrete areas of a solid support, wherein the nucleic acid probes are capable of hybridising to a codon of the template, adding the nucleic acid template or a sequence complementary thereto, to the array under conditions which allow for hybridisation, and observing the discrete areas of the support in which an hybridisation event has occurred.

### TECHNICAL FIELD OF THE INVENTION

The present invention relates to a method for obtaining structural information about an encoded molecule, especially an encoded molecule which is programmed by a template comprising codons that encode for chemical entities that have participated in the formation of the encoded molecule. The structural information may be used for deducting the entire structure of the encoded molecule or a part thereof. In addition, the obtained codon composition in the templates can be use for making new libraries of encoded molecules with desired properties.

### BACKGROUND

Array of oligonucleotides and polynucleotides have become an increasingly important tool in the bioscience industry. Array is currently used for nucleic acid sequencing, mutation analysis, and differential gene expression. A variety of different array technologies have been developed in order to meet the growing needs (for an overview see, Nature Genetics 32, December 2002).

Decoding techniques for nucleic acid templates, which encode molecules have previously been limited to amplification and/or cloning with subsequent sequencing, see e.g. WO 93/20242, WO 936/06121, WO 00/23458, WO 02/074929, and WO 02/103008.

The object of the present invention is to bring about a method which provides structural information of an encoded molecule in a fast and non-laborious manner.

### SUMMARY OF THE INVENTION

The present invention concerns a method for obtaining structural information about an encoded molecule, wherein the encoded molecule has been produced by a process comprising reacting a plurality of chemical entities, said chemical entities being coded for by codons on a nucleic acid template, the method comprising the steps of
i) providing an array comprising a plurality of single stranded nucleic acid probes immobilized in discrete areas of a solid support, wherein the nucleic acid probes are capable of hybridising to a codon of the template,
ii) adding the nucleic acid template or a sequence complementary thereto, to the array under conditions which allow for hybridisation,
iii) observing the discrete areas of the support in which an hybridisation event has occurred.

The term array or microarray generally refers to an ordered array of microscopic nucleic acid elements on a planar substrate. The microscopic nucleic acid elements forming a discrete spot are commonly referred to as features. Commercially available standard oligonucleotide microarrays may be used to prepare the array used in the method of the invention. Suitably, an oligonucleotide microarray is a device having a plurality of different single stranded oligonucleotide probes immobilized in discrete areas of a solid support. The discrete areas comprising immobilized single-stranded oligonucleotides may be referred to as spots for short.

The oligonucleotides immobilised in the spots may comprise any oligomer of nucleotides known in the art and in particular the nucleotides described below. Preferred nucleic acid probes are capable of forming a specific hybridisation with a complementing oligonucleotide segment. The complementing oligonucleotide segment is preferably at least one codon of a template. In some embodiments of the invention a probe of the array is able to hybridise to a two or more codons of a template. In another embodiment of the invention probes of the array is able to hybridize simultaneously to codons that have been located in the same template.

The solid support is preferable dimensional to add precision to the manufacturing and detection steps. Any specific dimension of the spotting area may be used. To adapt to the scanners usually used in the art, the solid support is usually of the dimension of a traditional 1×3-in microscope slide. The solid support is preferable flat, that is, the solid support has even parallel surfaces over a local region. The solid support should be uniform in the sense that irregularities preferably are avoid in the bulk of the support as well as in the surface coating or treatment. Preferably, the solid support is durable, i.e. a processed microarray should loose less than 10% of the annealed oligonucleotides over the assay duration, and inert, i.e. the solid support does not contribute any gain or loss of signal in the detection step. Usually, the solid support is a glass plate, a silicon or silicon-glass plate (e.g. a microchip), or a polymer plate.

Each spot on the solid support comprises the same nucleic acid probe, while at least one other spot comprises a different nucleic acid probe. The distance between each immobilized oligonucleotide on the spot is suitably 10 to 100 Å, and preferably between 10 and 50 Å to allow for optimized reaction kinetics and readily detection. The centre-to-centre spacing of the spots is suitable constant and in the range from 20 μm to 1000 μm and more preferred between 50 μm and 500 μm. The commercially available microarray Genflex (Affymetrix Inc.) may be adapted to be used in the present method, either directly or through the use of an adaptor oligonucleotide. The adaptor oligonucleotide has a sequence of nucleotides, which are complementary to the nucleic acid probe on the surface as well as the segment of the template it is intended to interact with. Thus, it will be clear to the skilled person that the term nucleic acid probe also can refer to a part of an adaptor oligonucleotide, which is able to hybridise to a segment of a template. Another type of available microarray is the inkjet printed arrays (Agilent Technologies Inc.) where the probes are synthesized directly on the array. Any type of microarray where probes are immobilized in discrete positions can be used in this invention.

The oligonucleotides immobilized on the solid support can be prepared in any convenient way. Usually, either a delivery approach or a synthesis approach is used. According to the delivery approach the oligonucleotides are synthesised, e.g. using the phosphoramidate method, and subsequent printed on the solid support, where the oligonucleotides are immobilized. The immobilization of preformed oligonucleotides may be performed utilizing any of a variety of attachment chemistries, such as (i) the formation of aminosilanes on a glass support and attaching the oligonucleotides thereto, (ii) the formation of an aldehyde surface on the solid support and reacting with an oligonucleotide comprising an amine, typically an aliphatic amine linker to form a covalent attachment, and (iii) the covalent attachment of an oligonucleotide carrying an anthraquinone to a polymer solid support as disclosed in WO 01/04129. The synthesis approach employs in situ synthesis of the oligonucleotides on the solid support using repeated addition of nucleotides until the final oligonucleotide eventually is formed. Usually, a method employing photo activation and masking is used.

It is preferred that the template is divided into coding regions or codons which codes for specific chemical entities. A codon is a sequence of nucleotides or a single nucleotide. The nucleotides are usually amplifiable and the nucleobases are selected from the natural nucleobases (adenine, guanine, uracil, thymine, and cytosine) and the backbone is selected from DNA and RNA, preferably DNA.

In the generation of a library, a codon of a single nucleotide will allow for the incorporation of four different chemical entities into the encoded molecule, using the four natural nucleobases (A, C, T, G). However, to obtain a higher diversity a codon in certain embodiments preferably comprises at least two and more preferred at least three nucleotides. Theoretically, this will provide for 4² and 4³, respectively, different chemical entities. The codons will usually not comprise more than 200 nucleotides. It is preferred to have codons with a sequence of 2 to 20 nucleotides, more preferred 4 to 15 nucleotides.

The term codon is used in some aspects of the invention not necessarily to defining the natural occurring codons that encodes for amino acids. Rather, the term codon refers to a designed sequence of nucleotides that code for a chemical entity, suitably other than α-amino acids.

The template will in general have at least two codons which are arranged in sequence, i.e. next to each other. Each of the codons may be separated by a framing sequence. Depending on the encoded molecule formed, the template may comprise further codons, such as 3, 4, 5, or more codons. Each of the further codons may be separated by a suitable framing sequence. Preferably, all or at least a majority of the codons of the template are arranged in sequence and each of the codons is separated from a neighbouring codon by a framing sequence. The framing sequence may have any appropriate number of nucleotides, e.g. 1 to 20. Alternatively, codons on the template may be designed with overlapping sequences.

Generally, it is preferred to have more than two codons on the template to allow for the synthesis of more diverse encoded molecules. In a preferred aspect of the invention the number of codons of the template is 2 to 100. Still more preferred are templates comprising 3 to 20 codons.

The framing sequence may serve various purposes. In one setup of the invention, the framing sequence identifies the position of a codon. Usually, the framing sequence either upstream or downstream of a codon comprises information which allows determination of the position of the codon.

The framing sequence may also or in addition provide for a region of high affinity. The high affinity region may ensure that the hybridisation of the template with the anti-codon will occur in frame. Moreover, the framing sequence may adjust the annealing temperature to a desired level.

A framing sequence with high affinity can be provided by incorporation of one or more nucleobases forming three hydrogen bonds to a cognate nucleobase. An example of a nucleobase having this property is guanine and cytosine. Alternatively, or in addition, the framing sequence may be subjected to back bone modification. Several back bone modifications provides for higher affinity, such as 2′-O-methyl substitution of the ribose moiety, peptide nucleic acids (PNA), and 2′-4′ O-methylene cyclisation of the ribose moiety, also referred to as LNA (Locked Nucleic Acid).

The size of the codon together with the framing sequence will determine the total length that will hybridize to the immobilized probe. This total length can vary dependent on the size of the codon and the framing sequences. Preferably the total length will be between 5 and 200 nucleotides and more preferably between 10 and 30 nucleotides.

The codon is preferably design to produce as many mismatches as possible between each codon. The mismatches will prevent cross-hybridization and make sure that only the right codon is hybridized to its probe on the array. The number of mismatches between the codons is determined both from the total diversity possible for a certain size (number of nucleotides) on the codon and the need of different codons. As a general rule, the longer the codon is the more mismatches is preferably to distinguish each individual codon during the hybridization to the probes on the microarray.

The template may comprise flanking regions around the codons. The flanking region can encompass a signal group, such a flourophor, a radio active group, to allow a direct detection of the presence of the complex or a label that may be detected, such as biotin. When the template comprises a biotin moiety, a hybridisation event can be observed by adding stained streptavidine, such as streptavidine-phycoerythrin conjugate. The template can also be labelled using Cyanine 3 and Cyanine 5b dye during amplification.

The flanking regions can also serve as priming sites for an amplification reaction, such as PCR. The template may in certain embodiments comprise an affinity region having the property of being able to hybridise to a building block.

It is to be understood that when the term template is used in the present description and claims, the template may be in the sense or the anti-sense format, i.e. the template can be a sequence of codons which actually codes for the molecule or can be a sequence complementary thereto. In some embodiments of the invention, the template is attached to the encoded molecule, when applied to the array, while in other embodiments the template is not attached to the encoded molecule. The former embodiment employing a template attached to the encoded molecule may be of advantage when it is the aim to detect whether a codon has survived a selection, while the latter embodiment using the template alone may be suitable when the template of a complex comprising an encoded molecule and the template that has encoded the molecule is amplified, e.g. by PCR. When applied to the array of single stranded nucleotide probes the template may be single stranded or double stranded. If the template is added to the array in a double stranded state, it is in general necessary to denaturate the double helix to obtain a hybridisation event with a probe.

The amount of template is normally lower than the amount of complementary probe to obtain a binding of the template to the probe below the saturation level. Using a shortage of template makes it possible to measure the relative amount of codon present. The measurement of the relative amount is of particular interest when more than one template having is present because it makes it possible to deduce whether two or more templates have the same codon.

When two samples of the template is prepared, a control sample and a test sample (labelled with Cyanine 3 and Cyanine 5, respectively for example), the amount of template is added at a high concentration to allow competition between the two samples. The ratio between the samples is then obtained from the signal of each dye and used as a measure of the relative amount of each codon in the samples.

The sequence of the template which anneals to the probe is in general more than 8 nucleotides to obtain a sufficient annealing temperature. The number of nucleotides which is involved in a hybridisation event is in suitably not above 200 because of the reaction kinetics. Preferably, the total annealing sequence of the template is between 8 and 50, most preferably between 10 and 25.

The encoded molecule is formed by a variety of reactants which have reacted with each other and/or a scaffold molecule. Optionally, this reaction product may be post-modified to obtain the final encoded molecule. The post-modification may involve the cleavage of one or more chemical bonds attaching the encoded molecule to the template in order more efficiently to display the encoded molecule.

The formation of an encoded molecule generally starts by a scaffold, i.e. a chemical unit having one or more reactive groups capable of forming a connection to another reactive group positioned on a chemical entity, thereby generating an addition to the original scaffold. A second chemical entity may react with a reactive group also appearing on the original scaffold or a reactive group incorporated by the first chemical entity. Further chemical entities maybe involved in the formation of the final reaction product. The formation of a connection between the chemical entity and the nascent encoded molecule may be mediated by a bridging molecule. As an example, if the nascent encoded molecule and the chemical entity both comprise an amine group a connection between these can be mediated by a dicarboxylic acid.

The encoded molecule may be attached directly to the template or through a suitable linking moiety. Furthermore, the encoded molecule may be linked to the template through a cleavable linker to release the encoded molecule at a point in time selected by the experimenter.

The chemical entities that are precursors for structural additions or eliminations of the encoded molecule may be attached to a building block prior to the participation in the formation of the reaction product leading the final encoded molecule. Besides the chemical entity, the building block generally comprises an anti-codon. In some embodiments the building block also comprise an affinity region providing for affinity towards the nascent complex.

Thus, the chemical entities are suitably mediated to the nascent encoded molecule by a building block, which further comprises an anti-codon. The anti-codon serves the function of transferring the genetic information of the building block in conjunction with the transfer of a chemical entity. The transfer of genetic information and chemical entity may occur in any order, however, it is important that a correspondence is maintained in the complex. The chemical entities are preferably reacted without enzymatic interaction. Notably, the reaction of the chemical entities is preferably not mediated by ribosomes or enzymes having similar activity.

According to certain aspects of the invention the genetic information of the anti-codon is transferred by specific hybridisation to a codon on the template. Other methods for transferring the genetic information of the anti-codon to the nascent complex are to anneal an oligonucleotide complementary to the anti-codon and attach this oligonucleotide to the complex, e.g. by ligation. A still further method involves transferring the genetic information of the anti-codon to the nascent complex using a polymerase and a mixture of dNTPs.

The chemical entity of the building block may in most cases be regarded as a precursor for the structural entity eventually incorporated into the encoded molecule. In other cases the chemical entity provides for the eliminations of chemical units of the nascent scaffold. Therefore, when it in the present application with claims is stated that a chemical entity is transferred to a nascent encoded molecule it is to be understood that not necessarily all the atoms of the original chemical entity is to be found in the eventually formed encoded molecule. Also, as a consequence of the reactions involved in the connection, the structure of the chemical entity can be changed when it appears on the nascent encoded molecule. Especially, the cleavage resulting in the release of the entity may generate a reactive group which in a subsequent step can participate in the formation of a connection between a nascent complex and a chemical entity.

The chemical entity of the building block comprises at least one reactive group capable of participating in a reaction which results in a connection between the chemical entity of the building block and another chemical entity or a scaffold associated with the nascent complex. The connection is facilitated by one or more reactive groups of the chemical entity. The number of reactive groups which appear on the chemical entity is suitably one to ten. A building block featuring only one reactive group is used i.a. in the end positions of polymers or scaffolds, whereas building blocks having two reactive groups are suitable for the formation of the body part of a polymer or scaffolds capable of being reacted further. One, two or more reactive groups intended for the formation of connections, are typically present on scaffolds.

The reactive group of the building block may be capable of forming a direct connection to a reactive group of the nascent complex or the reactive group of the building block may be capable of forming a connection to a reactive group of the nascent complex through a bridging fill-in group. It is to be understood that not all the atoms of a reactive group are necessarily maintained in the connection formed. Rather, the reactive groups are to be regarded as precursors for the structure of the connection.

The subsequent cleavage step to release the chemical entity from the building block can be performed in any appropriate way. In an aspect of the invention the cleavage involves usage of a reagent or and enzyme. The cleavage results in a transfer of the chemical entity to the nascent encoded molecule or in a transfer of the nascent encoded molecule to the chemical entity of the building block. In some cases it may be advantageous to introduce new chemical groups as a consequence of linker cleavage. The new chemical groups may be used for further reaction in a subsequent cycle, either directly or after having been activated. In other cases it is desirable that no trace of the linker remains after the cleavage.

In another aspect, the connection and the cleavage is conducted as a simultaneous reaction, i.e. either the chemical entity of the building block or the nascent encoded molecule is a leaving group of the reaction. In general, it is preferred to design the system such that the connection and the cleavage occur simultaneously because this will reduce the number of steps and the complexity. The simultaneous connection and cleavage can also be designed such that either no trace of the linker remains or such that a new chemical group for further reaction is introduced, as described above.

The attachment of the chemical entity to the building block, optionally via a suitable spacer can be at any entity available for attachment, e.g. the chemical entity can be attached to a nucleobase or the backbone. In general, it is preferred to attach the chemical entity at the phosphor of the internucleoside linkage or at the nucleobase. When the nucleobase is used for attachment of the chemical entity, the attachment point is usually at the 7 position of the purines or 7-deaza-purins or at the 5 position of pyrimidines. The nucleotide may be distanced from the reactive group of the chemical entity by a spacer moiety. The spacer may be designed such that the conformational spaced sampled by the reactive group is optimized for a reaction with the reactive group of the nascent encoded molecule.

In a preferred aspect of the invention a library or a sub-library of different templates is used. The complexes delivering the templates for the present method can be prepared in accordance with a variety of methods. Examples of these methods are depicted below and generally described in WO 02/103008 and WO 02/074929, the content if which is incorporated herein by reference in its entirety. Methods for generating libraries of complexes comprising a template linked to an encoded molecule may be referred to as Chemetics® herein below.

A first embodiment is based on the use of a polymerase to incorporate unnatural nucleotides as building blocks. Initially, a plurality of template oligonucleotides is provided. Subsequently primers are annealed to each of the templates and a polymerase is extending the primer using nucleotide derivatives which have appended chemical entities. Subsequent to or simultaneously with the incorporation of the nucleotide derivatives, the chemical entities are reacted to form a reaction product. The encoded molecule may be post-modified by cleaving some of the linking moieties to better present the encoded molecule.

Several possible reaction approaches for the chemical entities are apparent. First, the nucleotide derivatives can be incorporated and the chemical entities subsequently polymerised. In the event the chemical entities each carry two reactive groups, the chemical entities can be attached to adjacent chemical entities by a reaction of these reactive groups. Exemplary of the reactive groups are amine and carboxylic acid, which upon reaction form an amide bond. Adjacent chemical entities can also be linked together using a linking or bridging moiety. Exemplary of this approach is the linking of two chemical entities each bearing an amine group by a bi-carboxylic acid. Yet another approach is the use of a reactive group between a chemical entity and the nucleotide building block, such as an ester or a thioester group. An adjacent building block having a reactive group such as an amine may cleave the interspaced reactive group to obtain a linkage to the chemical entity, e.g. by an amide linking group.

A second embodiment for obtainment of complexes pertains to the use of hybridisation of building blocks to a template and reaction of chemical entities attached to the building blocks in order to obtain a reaction product. This approach comprises that templates are contacted with a plurality of building blocks, wherein each building block comprises an anti-codon and a chemical entity. The anti-codons are designed such that they recognise a sequence, i.e. a codon, on the template. Subsequent to the annealing of the anti-codon and the codon to each other a reaction of the chemical entity is effected.

The template may be associated with a scaffold. Building blocks bringing chemical entities in may be added sequentially or simultaneously and a reaction of the reactive group of the chemical entity may be effected at any time after the annealing of the building blocks to the template.

A third embodiment for the generation of a complex includes chemical or enzymatical ligation of building blocks when these are lined up on a template. Initially, templates are provided, each having one or more codons. The templates are contacted with building blocks comprising anti-codons linked to chemical entities. The two or more anti-codons annealed on a template are subsequently ligated to each other and a reaction of the chemical entities is effected to obtain a reaction product.

A fourth embodiment makes use of the extension by a polymerase of an affinity sequence of the nascent complex to transfer the anti-codon of a building block to the nascent complex. The method implies that a nascent complex comprising a scaffold and an affinity region is annealed to a building block comprising a region complementary to the affinity section.

Subsequently the anti-codon region of the building block is transferred to the nascent complex by a polymerase. The transfer of the chemical entity may be transferred prior to, simultaneously with or subsequent to the transfer of the anti-codon.

Thus, the codons are either pre-made in one or more templates before the encoded molecules are generated or the codons is combined simultaneously together with the encoded molecules. The codons will possess at least to functions, viz. encoding for chemical entities and identification of the chemical entities.

After or simultaneously with the formation of the reaction product some of the linkers to the template may be cleaved, however at least one linker must be maintained to provide for the complex.

In one aspect of the invention, the library of the complexes as such is added to the oligonucleotide microarray under hybridisation conditions in order for each template to anneal to a cognate probe on the microarray. However, prior to the annealing step of the invention, it is preferred to subject the library to a condition, wherein an encoded molecule or a sub-library of encoded molecules having a predetermined property has been partitioned from the remained of the library. The partition step may be referred to as a selection or a screen, as appropriate, and includes the screening of the library for encoded molecules having predetermined desirable characteristics. Predetermined desirable characteristics can include binding to a target, catalytically changing the target, chemically reacting with a target in a manner which alters/modifies the target or the functional activity of the target, and covalently attaching to the target as in a suicide inhibitor.

In another aspect of the invention, the library of complexes is subjected to partitioning against different targets. After the partition step the templates are amplified and labelled using different dyes (for example, Cyanine 3 and Cyanine 5). Then the two amplified samples is mixed and analysed on the array to identify the codons. The ratio between the two samples obtained in this example can be used to elucidate the specificity of the partitioned complexes between various targets. This comparative strategy can be used for any targets or reference samples.

The target can be any compound of interest. The target can be a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, cell, tissue, etc. without limitation. Particularly preferred targets include, but are not limited to, angiotensin converting enzyme, renin, cyclooxygenase, 5-lipoxygenase, IIL-10 converting enzyme, cytokine receptors, PDGF receptor, type II inosine monophosphate dehydrogenase, β-lactamases, and fungal cytochrome P-450. Targets can include, but are not limited to, bradykinin, neutrophil elastase, the HIV proteins, including tat, rev, gag, int, RT, nucleocapsid etc., VEGF, bFGF, TGFβ, KGF, PDGF, thrombin, theophylline, caffeine, substance P, IgE, sPLA2, red blood cells, glioblastomas, fibrin clots, PBMCs, hCG, lectins, selectins, cytokines, ICP4, complement proteins, etc.

Encoded molecules having predetermined desirable characteristics can be partitioned away from the rest of the library while still attached to the nucleic acid template by various methods known to one of ordinary skill in the art. In one embodiment of the invention the desirable products are partitioned away from the entire library without chemical degradation of the attached nucleic acid template such that the templates are amplifiable. The templates may then be amplified, either still attached to the desirable encoded molecule or after separation from the desirable encoded molecule.

In the most preferred embodiment, the desirable encoded molecule acts on the target without any interaction between the template attached to the desirable encoded molecule and the target. In one embodiment, the bound complex-target aggregate can be partitioned from unbound complexes by a number of methods. The methods include nitrocellulose filter binding, column chromatography, filtration, affinity chromatography, centrifugation, beads, plastic surfaces, and other well known methods.

Briefly, the library of complexes is subjected to the partitioning step, which may include contact between the library and a column onto which the target is immobilised. Templates associated with undesirable encoded molecules, i.e. encoded molecules not bound to the target under the stringency conditions used, will pass through the column. Additional undesirable encoded molecules (e.g. encoded molecules which cross-react with other targets) may be removed by counter-selection methods. Desirable complexes are bound to the column and can be eluted by changing the conditions of the column (e.g., salt, pH, surfactant, etc.) or the template associated with the desirable encoded molecule can be cleaved off and eluted directly. The elution can also be performed using a known ligand that displaces the target-bound complexes.

Additionally, chemical compounds which react with a target can be separated from those products that do not react with the target. In one example, a chemical compound which covalently attaches to the target (such as a suicide inhibitor) can be washed under very stringent conditions. The resulting complex can then be treated with various proteinase, reducing agents or other suitable reagents to cleave a linker and liberate the nucleic acids which are associated with the desirable chemical compound. The liberated nucleic acids can be amplified.

In another example, the predetermined characteristic of the desirable product is the ability of the product to transfer a chemical group (such as acyl transfer) to the target and thereby inactivate the target. One could have a product library where all of the products have a thioester chemical group. Upon contact with the target, the desirable products will transfer the chemical group to the target concomitantly changing the desirable product from a thioester to a thiol. Therefore, a partitioning method which would identify products that are now thiols (rather than thioesters) will enable the selection of the desirable products and amplification of the nucleic acid associated therewith.

It can be envisaged that the codons in the templates are physically separated from each other before analysed on the array. This can be performed using for example restriction enzymes. A specific cut site can be engineered in the sequence between the codon to allow this. The separation of the codons can also be performed using a more random approach to obtain small fragment of the template. This can be accomplished using sheering of DNasel treatment. Other enzymes that cut double or single stranded DNA can also be used. The will produce small fragments of the template that contains the codons. The physically separation of the codons before array analysis might be an advantage to prevent competition between binding of the same template to multiple probes immobilized on the array.

There are other partitioning and screening processes which are compatible with this invention that are known to one of ordinary skill in the art. In one embodiment, the products can be fractionated by a number of common methods and then each fraction is then assayed for activity. The fractionization methods can include size, pH, hydrophobicity, etc.

Inherent in the present method is the selection of encoded molecules on the basis of a desired function; this can be extended to the selection of molecules with a desired function and specificity. Specificity can be required during the selection process by first extracting templates of chemical compounds which are capable of interacting with a non-desired “target” (negative selection, or counter-selection), followed by positive selection with the desired target. As an example, inhibitors of fungal cytochrome P-450 are known to cross-react to some extent with mammalian cytochrome P-450 (resulting in serious side effects). Highly specific inhibitors of the fungal cytochrome could be selected from a library by first removing those products capable of interacting with the mammalian cytochrome, followed by retention of the remaining products which are capable of interacting with the fungal cytochrome.

Ideally, the array comprises a nucleic acid probe for each template. However, this approach is not always feasible for large libraries. Therefore, according to a preferred aspect of the present invention only a fraction of the entire number of codons for each template is matched by a cognate probe. Accordingly, in a preferred aspect, the probe of the array may be hybridised to all but one of the codon of a template or less. As an example, when the template comprises 4 codons, the probe may hybridise to 1, 2, or 3 codons.

The template or library of templates may be added to the array while attached to the encoded molecule or the template of the complex may be added as a component detached from the encoded molecule, e.g. as a PCR fragment. In general, it is preferred to amplify the templates before the addition to the array in order to obtain a more sensitive measurement. Furthermore, the amplification may introduce a label which at a later stage may serve to measure a hybridisation event. As an example, biotin may be introduced by incorporating it in a primer which is extended over the template or the complementing sequence thereof thereby obtaining a template (or its complementary sequence) labelled with biotin. In a step following the hybridisation between probes of the array with any templates, any possible hybridisation event may be observed by the addition of stained streptavidine. A variety of possible labelling methods are known to the skilled person, including direct detection, e.g. using cy5, or indirectly using an epitop recognised by a suitable antibody, which may be linked to a flourophor or an enzyme capable of converting a substrate to a detectable product.

The hybridisation conditions may be appropriately adjusted by a person skilled in the art taking into account the number and kind of nucleobases that participate in the formation of the hybrid.

It is within the capability of the skilled person in the art to construct the desired design of an oligonucleotide. When a specific annealing temperature is desired it is a standard procedure to suggest appropriate compositions of nucleic acid monomers and the length thereof. The construction of an appropriate design may be assisted by software, such as Vector NTI Suite or the public database at the internet address http://www.nwfsc.noaa.gov/protocols/oligoTMcalc.html.

The conditions which allow hybridisation of the templates and probes are influenced by a number of factors including temperature, salt concentration, type of buffer, and acidity. It is within the capabilities of the person skilled in the art to select appropriate conditions to ensure that the contacting between the templates and the probes is performed at hybridisation conditions. The temperature at which two single stranded oligonucleotides forms a duplex is referred to as the annealing temperature or the melting temperature. The melting curve is usually not sharp indicating that the annealing occurs over a temperature range. The second derivative of the melting curve is used herein to indicate the annealing temperature.

The measurement of a hybridisation event may be conducted by various methods known in the art. In the event the label emits lights, the presence or absence of a hybridisation event may be measured in a scanner, e.g. a confocal scanner. The scanner may be connected with computer software, which is able to quantify the amount of lights measured. The amount of light measured correlates with the amount of template annealed to the probes. Thus, it is possible to measure not only the presence or absence of one or more codons of a template; it is also possible to measure the relative amount of the codons in one or more templates.

The method according to the present invention may be applied for only a single codon or multiple codons of a template. When a nucleic acid probe of the array is designed only to measure a single codon, the information from the observation of the absence or the presence of a hybridisation event may be used for a variety of purposes. The information that a codon is present in a pool of templates, may be used to check whether a selection actually has occurred, if templates of library is added to the array before and after a selection. Moreover, the detection of single codons can be used for adjusting the selection pressure, e.g. by adjusting the pH, ion strength, temperature etc. to a desired level. If a further selection is required, a limited pool of complexes may be produced by using only such building blocks which comprise anti-codons and chemical entities which in encoded for by the template. In the event a certain codon is extensively present in the pool of templates, this may be significant information for establishing a structure-activity-relationship (SAR).

Further useful information about a certain codon may be gathered by detecting the codon together with a framing sequence identifying the position in the reaction history of the chemical entity corresponding to said codon.

As an example, if a library of complexes is prepared from 100 building blocks and the four reactions, i.e. each template comprises 4 codons, the library size is 10⁸. Fore most practical uses 10⁸ is in the excess of what is possible to detect on an array, especially if multiple determinations for each template are considered necessary to obtain a high accuracy. However, an array of just 100 probes complementary to the 100 codons will reveal important information prior to or subsequent to a selection. In the event a framing sequence is detected together with the codon an array of 400 probes is needed.

In one embodiment of the invention two codons are detected simultaneously, i.e. the probes of the array are designed to anneal to two neighbouring codons of a template. Two codons adjacent to each other provide information not solely of the chemical entities which have participated in the formation of the encoded molecule. Also the order in which the two chemical entities have reacted is obtained. Further information may be provided by measuring one or more framing sequences together with the codons, because information on the position in the synthesis history may be coded for by the framing sequence. As an example a template of three codons may be identifier by just two probes using centre codon as the bridge for coupling the two flanking codons to each other. Thus, an otherwise large array may be reduced considerable in size, while still obtaining the same valuable information.

When a library or sub-library of templates is analysed on a two-codon detecting array a structure-activity-relationship may be deduced, especially when a preceding PCR amplification has been conducted. A PCR amplification will amplify the templates in a linear fashion, i.e. if a template is present in relatively high number following a selection, this template will eventually appear in a high concentration. Thus, if a complex comprises an encoded molecule being a good binder, i.e. having a high affinity to the target, the template of the complex will be amplified to a relative higher concentration compared to an encoded molecule having less affinity towards the target. On the array an encoded molecule having a high affinity displays itself through the codon and thus provides the experimenter of the structure of chemical entities leading to a high activity. The information on good binders may be used in traditional rational design for developing new compounds for screening or may be used for including building blocks for a subsequent generation of a second library.

After the complexes have been partitioned and the specific codons have been identified on the microarray, the information can be used to design optimized libraries including chemical entities based on both the selection data and the chemical structure. The microarray analysis will first of all detect which chemical entities pass the partitioning step. Secondly, the relative intensity on the microarray will reflects the relative binding affinity of the chemical entities. Finally, the structures of the chemical entities are directly identified due to the position of the probes on the array. For instance, chemical entities that are strongly selected in a partitioning process but possess some unfavourable chemical structure can be excluded in the next generation of library. Similarly, chemical entities that are weekly selected in a partitioning process but possess some favourable chemical structure can be included in the next generation of library. Thus, the next generation library design can be based both on a rational choice of chemical entities with lead-like structures and the selection pressure detected on the microarray.

While the present invention has been exemplified for a one- or two-codon detecting array it should be apparent for the person skilled in the art that the same methods may be applied for probes detecting higher numbers of codon, e.g. 3, 4, or 5 codons. In addition, a combination of microarrays detecting various numbers of codons can also be used to obtain complementing data using structure-activity relationship analysis for instance.

Nucleotides

The nucleotides used in the present invention may be linked together in an oligonucleotide. Each nucleotide monomer is normally composed of two parts, namely a nucleobase moiety, and a backbone. The back bone may in some cases be subdivided into a sugar moiety and an internucleoside linker.

The nucleobase moiety may be selected among naturally occurring nucleobases as well as non-naturally occurring nucleobases. Thus, “nucleobase” includes not only the known purine and pyrimidine hetero-cycles, but also heterocyclic analogues and tautomers thereof. Illustrative examples of nucleobases are adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diamino-purine, 5-methylcytosine, 5-(C³-C⁶)-alkynylcytosine, 5-fluorouracil, 5-bromouracil, pseudoisocytosine, 2-hydroxy-5-methyl-4-triazolopyridine, isocytosine, isoguanine, inosine and the “non-naturally occurring” nucleobases described in Benner et al., U.S. Pat. No. 5,432,272. The term “nucleobase” is intended to cover these examples as well as analogues and tautomers thereof. Especially interesting nucleobases are adenine, guanine, thymine, cytosine, 5-methylcytosine, and uracil, which are considered as the naturally occurring nucleobases in relation to therapeutic and diagnostic application in humans.

Examples of suitable specific pairs of nucleobases are shown below:

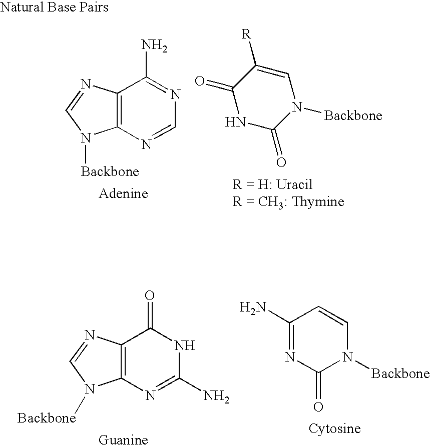

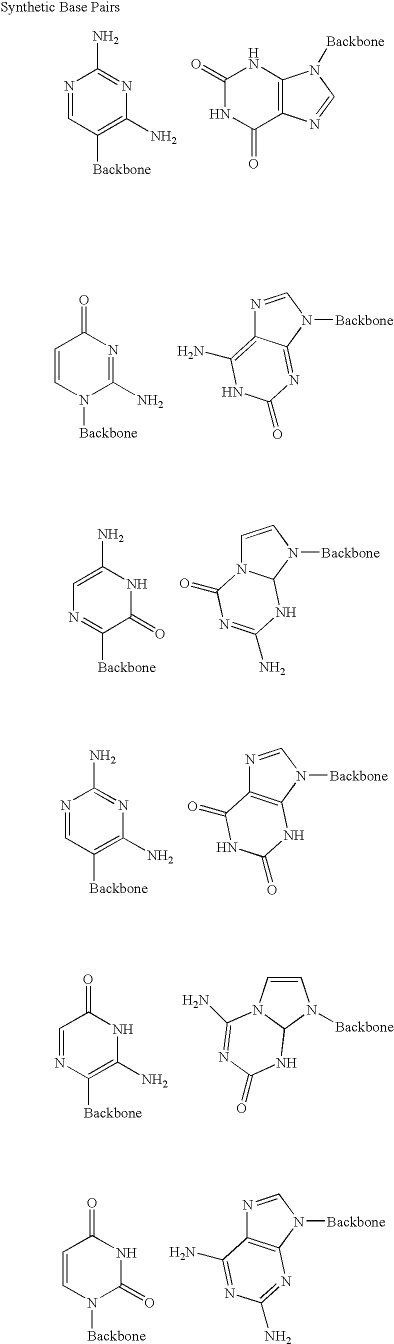

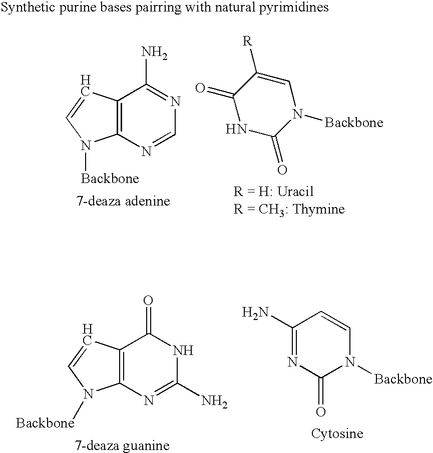

Suitable examples of backbone units are shown below (B denotes a nucleobase):

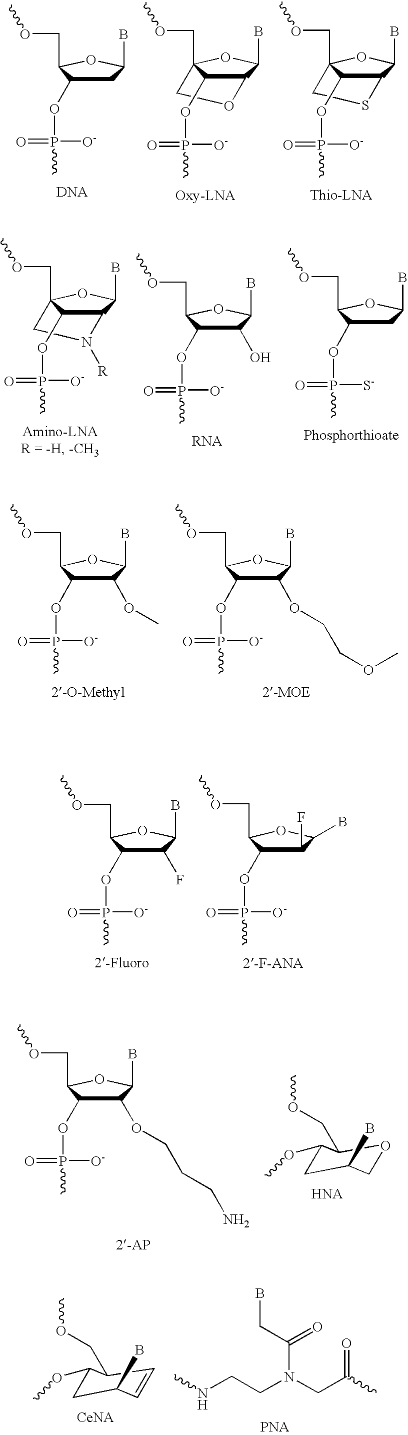

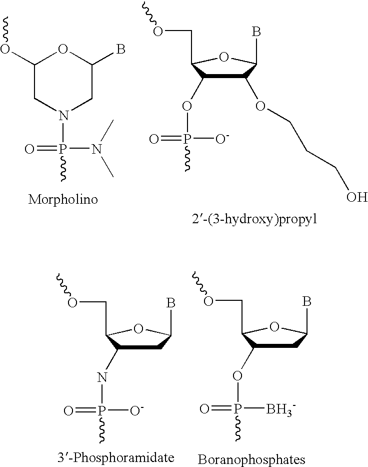

The sugar moiety of the backbone is suitably a pentose but may be the appropriate part of an PNA or a six-member ring. Suitable examples of possible pentoses include ribose, 2′-deoxyribose, 2′-O-methyl-ribose, 2′-flour-ribose, and 2′-4′-O-methylene-ribose (LNA). Suitably the nucleobase is attached to the 1′ position of the pentose entity.

An internucleoside linker connects the 3′ end of preceding monomer to a 5′ end of a succeeding monomer when the sugar moiety of the backbone is a pentose, like ribose or 2-deoxyribose. The internucleoside linkage may be the natural occurring phospodiester linkage or a derivative thereof. Examples of such derivatives include phosphorothioate, methylphosphonate, phosphoramidate, phosphotriester, and phosphodithioate. Furthermore, the internucleoside linker can be any of a number of non-phosphorous-containing linkers known in the art.

Preferred nucleic acid monomers include naturally occurring nucleosides forming part of the DNA as well as the RNA family connected through phosphodiester linkages. The members of the DNA family include deoxyadenosine, deoxyguanosine, deoxythymidine, and deoxycytidine. The members of the RNA family include adenosine, guanosine, uridine, cytidine, and inosine. Inosine is a non-specific pairing nucleoside and may be used as universal base as discussed above because inosine can pair nearly isoenergetically with A, T, and C.

### BRIEF DESCRIPTION OF THE FIGURES

FIG. 1 shows the principle of a preferred embodiment of the invention.

FIG. 2 shows the identification and characterization of encoded molecules in array format.

FIG. 3 shows enrichment of templates mediated by the encoded molecules.

FIG. 4 shows the principle of decoding using an array FIG. 5 shows the detection of single codons of templates.

FIG. 6 shows the detection of codon pairs of templates.

FIG. 7 shows the detection of codon pairs at specific codon positions.

FIG. 8 shows the detection of single codons of templates after the separation of the individual codons.

### DETAILED DESCRIPTION OF THE INVENTION

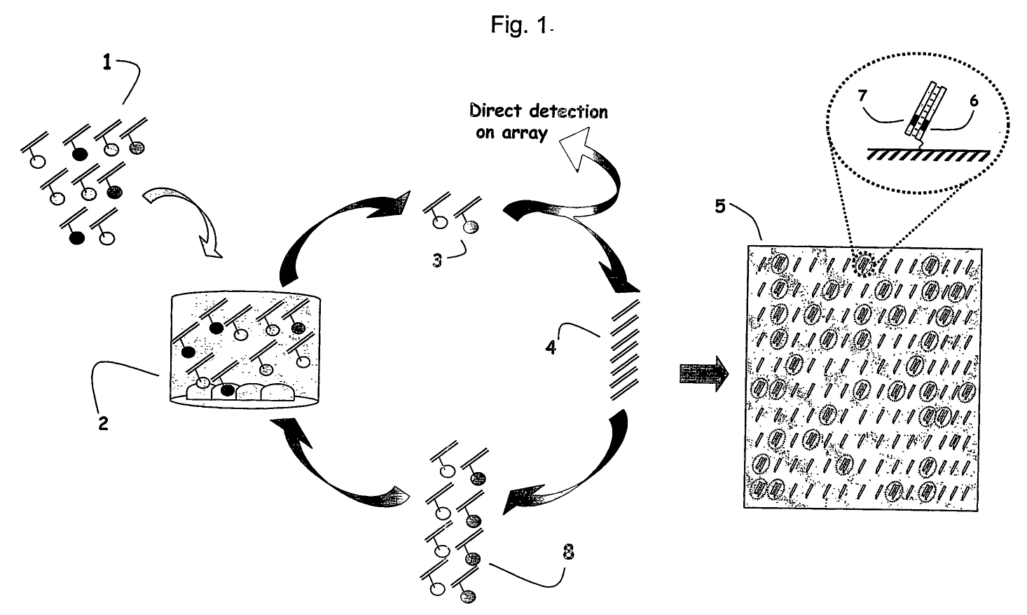

In FIG. 1, an over-all scheme of a preferred embodiment is illustrated. Initially, a library of complexes (1), each comprising an encoded molecule and a template is provided. The library may be produced by a number of approaches, including the methods disclosed in WO 02174929 A2 and WO 02/103008 A2. The library of complexes is subjected to a selection (2). The selection includes the immobilisation of targets to a support and subsequent exposure of the library to the targets. Some of the encoded molecules may be bound to the target, while other will be maintained in the solution. The non-bound complexes of the library are eluated away for the immobilized target. The complexes bound to the target may be recovered by increasing the stringency of the media of the target, e.g. by increasing the temperature, the salt concentration, pH etc. The recovered complexes are a sub-library (3) of the initial library and may be contacted directly with an array in order to analyse the identity of the codons. Preferably, the templates of the complexes are amplified, e.g. by applying PCR, to obtain a sufficient quantity of the template for a sensitive detection on the array. The PCR amplicons (4) may be applied as such to the array (5) or either the sense or anti sense strand of the amplicons may be digested in order to obtain a single stranded template or a sequence complementary thereto. The PCR process may introduce a suitable label. Each probe (6) of the array presents its nucleic acid sequence. If a complementing sequence, i.e. a template (7) is present this sequence will hybridise to the probe and form a double helix structure. The label is used to detect the spots of the array in which a hybridisation has occurred between a probe and a template, thereby identifying the presence of one or more codons of the templates in the sub-library.

The information obtained by the analysis of the codons on the array may be used for a variety of purposes. The information may be give rise to a change in the selection process if to many or too few templates (containing various codons) are detected following the selection. The change in the selection process may include a higher or lower stringency condition during the binding of the encoded molecule to the immobilized target. The information may also be used for generating a SAR (structure-activity-relationship), especially when the relative abundance of codons is possible. The SAR information can be used for the generation of a further library said further library being a sub-library of the initial library of complexes or comprising structure elements from chemical entities in the encoded molecule not previously used. Further, the information possible to deduce from the array analysis may be used to establish the synthesis history of the encoded molecule. The synthesis history may be established in its entirety or partially and includes information about the chemical entities which have participated in the formation of the encoded molecule, the process condition used during the synthesis of each step, and the point in time of the formation of the encoded molecule in which a particular chemical entity has entered the reaction pathway.

The amplified templates may be used for generating a new library (8) enriched with encoded molecules, which binds to the target. The enriched library may be subjected to a new screening process using the same target (2), however applying more stringent conditions during the binding step.

The general principle shown in FIG. 1 may be used in on or more rounds, i.e. the selection, amplification and generation steps may be conducted one or more times. The analysis of the templates on the array need not be employed in each round. In some embodiments the array analysis is only conducted in the last round in order to detect the best binders in the library. In other embodiments the analysis of the templates on an array is conducted in the initial rounds to obtain a SAR which may be used to identify chemical entities of importance for the formation of a good binding encoded molecule.

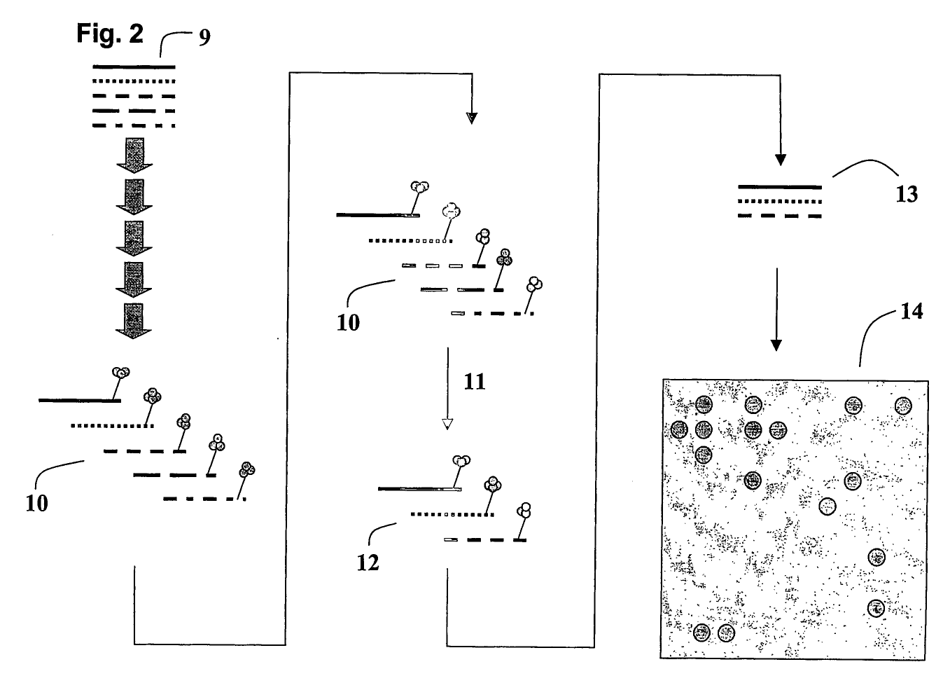

FIG. 2 discloses a method for identification and characterisation of hits from a selection process. Initially, genetic information (9) is provided either as entire templates, as fractions thereof or as anti-codons. Through various techniques, commonly known Chemetics®, the genetic information is transformed into a library of complexes (10) comprising an encoded molecule linked to the template which codes for the synthetic history thereof. The library is subjected to a selection process to form a sub-library (12) comprising ligands that binds to the target. Subsequently, the templates of the sub-library is amplified to form a pool of PCR products (13), which comprises the genetic information of the templates, i.e. the codons of the templates. The PCR product is added to an array (14) of single stranded polynucleotides complementary to one or more codons of the templates, thereby identifying the hits of the library. Usually, the amplification process involves the incorporation of a measurable label.

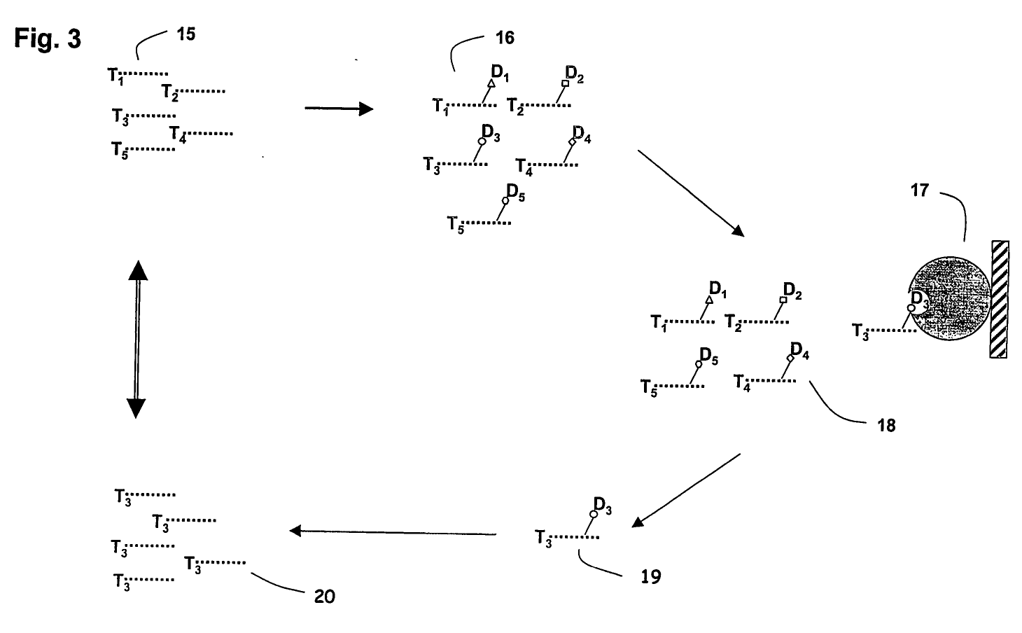

FIG. 3 shows a method for enrichment of templates of complexes which have affinity for a target. Initially a pool of genetic information (15) is provided. The genetic information is transformed into a library of complexes (16) according to any Chemetics® method. The library is subjected to enrichment in respect of complexes which bind to an immobilized target (17). The non-binding complexes (18) are eluted away from the device comprising the immobilized target, such a column. Subsequently, the binding complex (19) is eluted from the immobilized target using stringent conditions. The sub-library formed after the selection process is subjected to amplification in order to form more copies of the individual complexes in the sub-library. The amplification is in general divided into to separate steps. First, the templates of the enriched library are amplified, e.g. using PCR, to produce a pool of PCR fragments (20), which comprises the genetic information of the templates. Second, the pool of amplified templates is transformed into complexes (15) using a templating method, such as Chemetics®. The concentration of the selected templates directly corresponds to the concentration of the selected encoded molecules, i.e. the encoded molecules binding with a higher affinity to the target will be represented in the sub-library in a higher concentration compared to both non-binders and binding molecules with lower affinity.

The enriched library of the complexes may be subjected to a further selection process as depicted above.

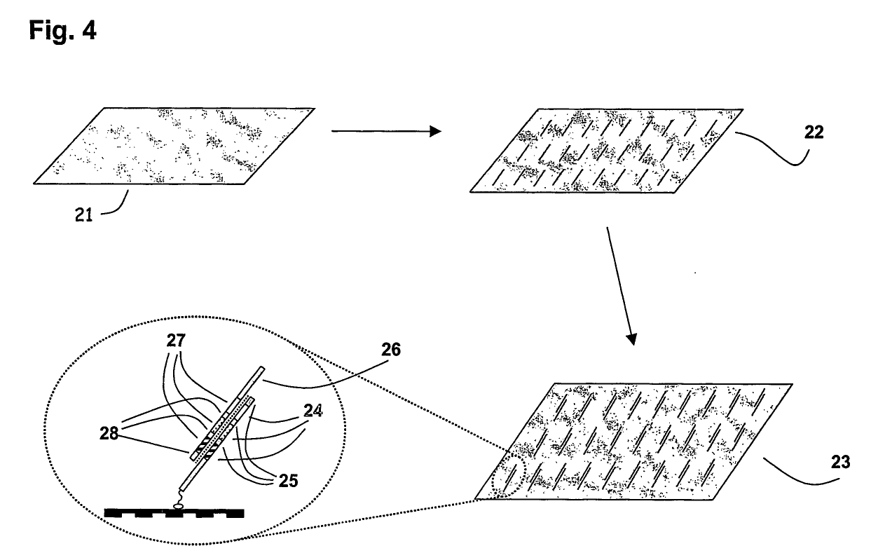

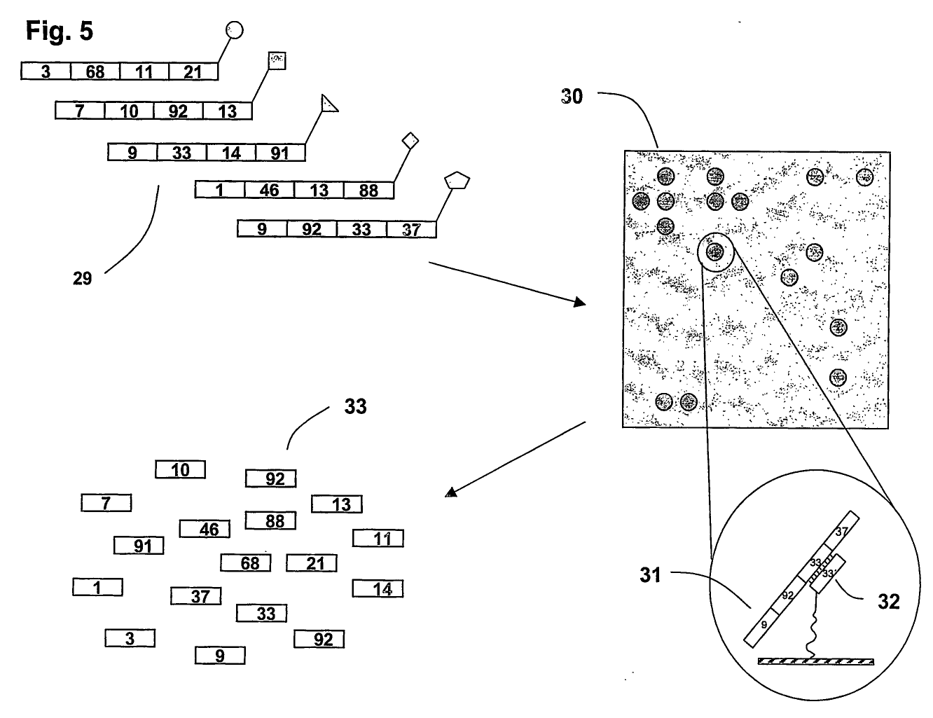

FIG. 4 shows the principle of using arrays for detecting codons of templates. A solid support (21), e.g. a glass slide, is initially provided. Probes of oligonucleotides are immobilized on the solid support (22) by the delivery approach or the synthesis approach as disclosed in for instance Schena, Mark: Microarray analysis (2003). Subsequently, the PCR fragments of a selected library of complexes are added to the single stranded array at conditions which allow the double stranded PCR fragments to denaturise and a hybridisation to a cognate probe of the array (23). The probe of the array is illustrated with three anti-codons (24) separated with complementing framing sequences (25). The template (26) hybridised to the probe comprises three codons (27) recognised by the anti-codons of the probe and the three framing sequences (28) recognised by the complementing framing sequences. Each of the codons is linked to a framing sequence identifying the position of the codon on the template. The illustrated template can be fully characterized by the hybridising the probe because the spatial position of the probe will be informative of the codons. A determination of the codons and their relative position will map the synthesis history of the encoded molecule, i.e. the codons will provide information on the chemical entities which have participated in the formation of the encoded molecule and of the order in which the chemical entities have reacted.

FIG. 5 shows an array detection system in which a single codon is detected. Initially a library of selected complexes (29), i.e. complexes comprised of the initial library which display a certain property, is provided as disclosed above. The initial library of complexes is prepared from e.g. 100 codons and templates having 4 codons in sequence, which theoretical gives a library of 10⁸ complexes. The selected complexes are subjected to amplification to amplify the templates of the selected complexes and the amplification products are added to an array (30). The array (30) comprises probes (31) complementary to each of the codons of the templates (32). At hybridisation conditions the PCR products of the templates are annealed to the cognate probes of the array and in a suitable scanner the spatial position of the annealed probes are detected to elucidate the codons (33) of the template. The quantity of each codon may be measured to find codons abundant in more than one template and/or codons leading to encoded molecules with high affinity. The information may be used for decoding of the encoded molecule of the complexes displaying the desired property or the information may be used for selection of building blocks which is to be added in a next round of library formation.

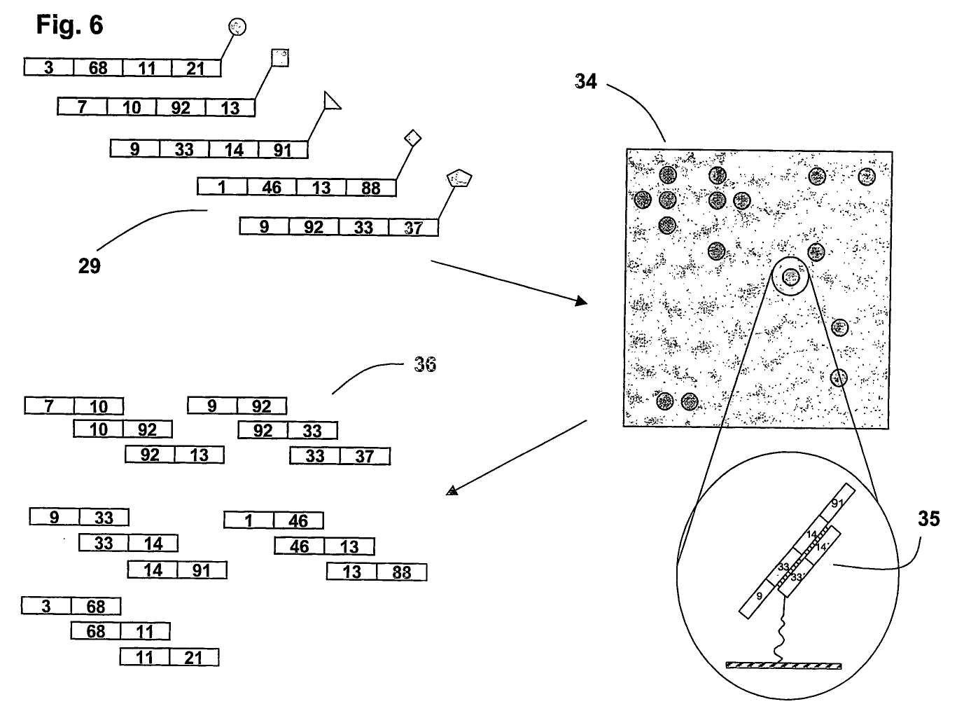

FIG. 6 discloses an array detection system for establishing codons pairs, i.e. codons in the vicinity of each other. Initially (as shown in this example) a library of complexes is prepared from 100 different codons deposited on a template in a sequence of four, making the total amount of combinations possible 10⁸. The initial library is subjected to a condition in order to select a sub-library (29) displaying a desired property. The templates of the sub-library are amplified by a PCR reaction and the reaction product is added under hybridisation conditions to an array (34). The array is designed with probes (35) capable of detecting two codons at a time. To cover all possible combinations of a library based on 100 different codons 10⁴ probes are needed, which is practically feasible with the current technology.

The detection of the codons may be conducted quantitatively, i.e. the relative abundance of each of the codon pairs may be determined. The detection on the array may be used to reconstruct the selected templates (36) as three overlapping codon pair detections depict the entire template. In the event the same codon pair appears on more than one template, the information on the relative abundance of each codon pair maybe used to decipher the sequence of codons of the selected templates as it can be assumed that each codon pair of the same template appears in the same amounts in the PCR products added to the array.

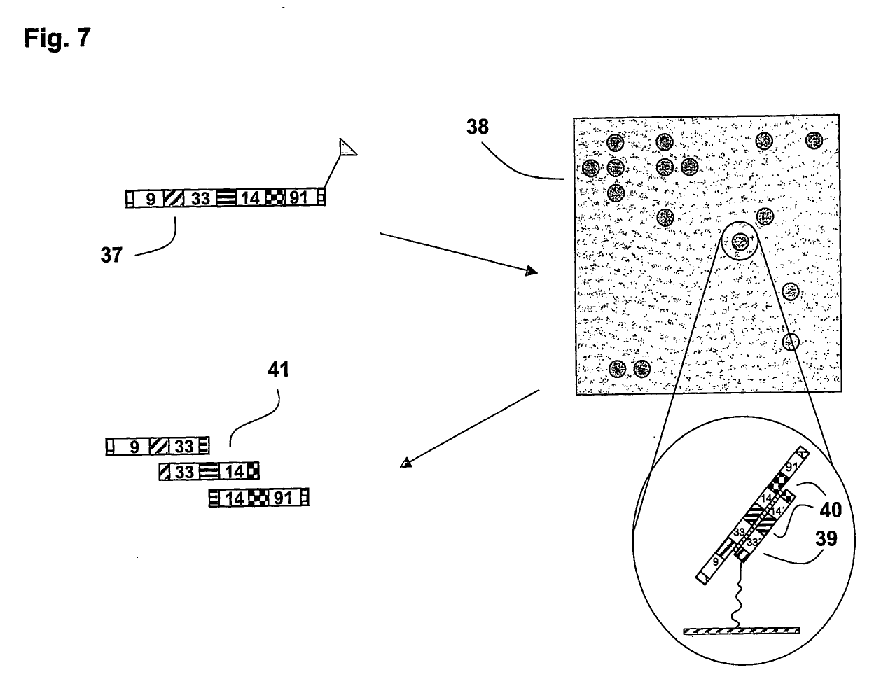

FIG. 7 discloses an array for detecting codon pairs at specific codon positions. Initially, a library of complexes comprising templates with framing sequences is provided. The framing sequence is specific for each position of the codons on the template. Four times more probes on the microarray is needed per each codon if the position of the codons also should be detected in the analysis which is practically feasible with current technology. The position is detected due to the framing sequences next to each codon. The initial library is subjected to a selection process to isolate complexes (37) having a desired property. The selected complexes are amplified by a PCR reaction and the reaction products are added to an array (38). The array comprises probes capable of detecting codon pairs as wells as the framing sequences (40) between the codons. The framing sequence determines the position of the codon in the reaction history, i.e. it is possible to deduct which chemical entity that reacted at which point in time of the synthesis history of the encoded molecule, thus making it possible to reconstruct the structure of the encoded molecule.

The detection of the codon pairs may be conducted quantitatively, i.e. the relative abundance of each of the codon pairs may be determined. The detection on the array may be used to reconstruct the selected templates (41) as three overlapping codon pair detections depict the entire template. In the event the same codon pair appears on more than one template, the information on the relative abundance of each codon pair maybe used to decipher the sequence of codons of the selected templates as it can be assumed that each codon pair of the same template appears in the same amounts in the PCR products added to the array.

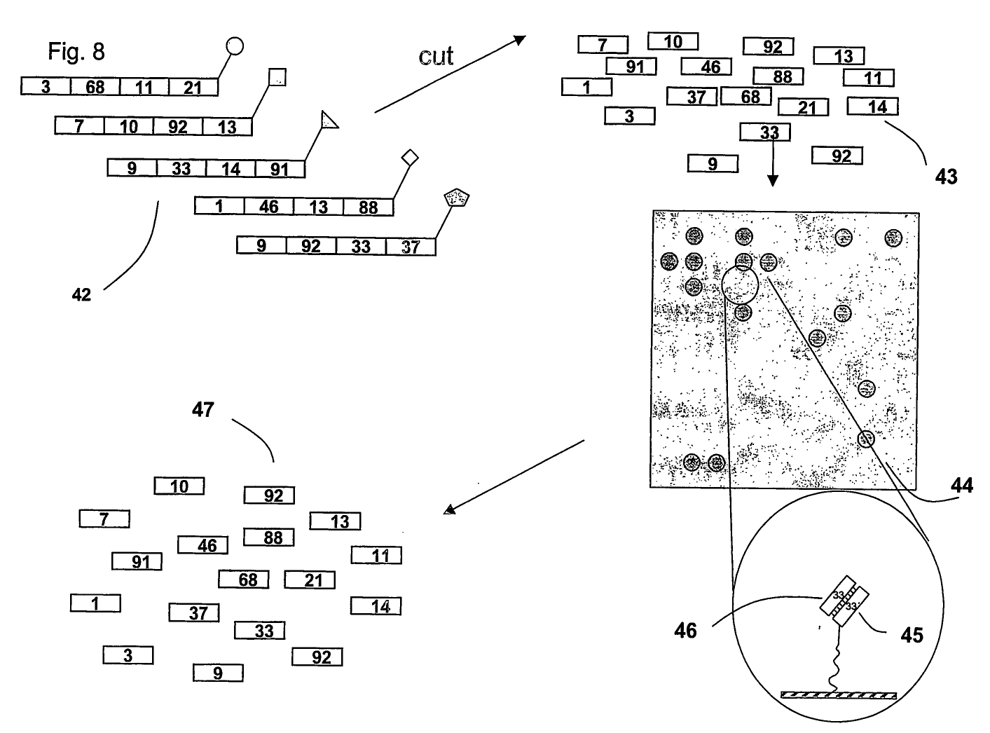

FIG. 8. shows an array detection system in which a single codon is detected. Initially a library of selected complexes (42), i.e. complexes comprised of the initial library which display a certain property, is provided as disclosed above. The initial library of complexes is prepared from e.g. 100 codons and templates having 4 codons in sequence, which theoretical gives a library of 10⁸ complexes. The selected complexes are subjected to amplification to amplify the templates of the selected complexes and the amplification products are treated with suitable reagents to cut between the individual codons (43). The individual codon is the applied to the array. The array (44) comprises probes (45) complementary to each of the codons of the templates (46). At hybridisation conditions the PCR products of the templates are annealed to the cognate probes of the array and in a suitable scanner the spatial position of the the annealed probes are detected to elucidate the codons (47) of the template. The quantity of each codon may be measured to find codons abundant in more than one template and/or codons leading to encoded molecules with high affinity. The information may be used for decoding of the encoded molecule of the complexes displaying the desired property or the information may be used for selection of building blocks which is to be added in a next round of library formation.

### EXAMPLES

### Example 1

### Detection of Single Codons

This example shows the possibility to determine the exact location of specific codons in template molecules.

Six adaptors with the same anti-codon in all three positions were designed (underlined), only the framing regions were different (Bold). All the adaptors contain a probe binding sequence (20 nucleotides) that allow discrete binding on the microarray. Adaptors harbouring one to three deletions in the spacing region were used as negative controls to ensure that only the framing region is responsible for the hybridization of the template. Thus, the negative controls contain another framing sequence. The template oligonucleotide harbours the complementing codon sequence and the position directing framing regions.

Adaptor oligonucleotides
3′CTCATCGGAAGGGCTCGTAACGGTGGGTTTGGGGGCTGGGTTTGGGGCGTGGGTTT
GGGCGG-5′
3′TTTGGTAGCTGAGTGCCCTAGGCTGGGTTTGGGCGGTGGGTTTGGGGGCTGGGTTT
GGGGCG-5′
3′TAACTGGTTTGACGCCACGCGCGTGGGTTTGGGGCGTGGGTTTGGGCGGTGGGTTT
GGGGGC-5′
3′TAATTGAGCTGACGGCGCACGGCTGGGTTTGGGCGTGGGTTTGGGGCTGGGTTTGG
GGCG-5′
3′TGTTGCTACTCTGGCCCGAGGCTGGGTTTGGGCTGGGTTTGGGCTGGGTTTGGGGC
G-5′
3′ACGGGATAACAACGCAGCCTGGCTGGGTTTGGGTGGGTTTGGGTGGGTTTGG-
GGCG-5′

Template Oligonucleotide

Biotin-5′GCCACCCAAACCCCCG

GenFlex hybridisation and scanning. Prior to hybridization, the Adaptor mix (100 μM final concentration for each of the adaptor oligonucleotides) in a hybridization buffer (100 mM MES, 1 M NaCl, 20 mM EDTA, 0.01% Tween 20, 1× Denhardt's), was heated to 95° C. for 5 min and subsequently cooled and maintained at 40° C. for 5 min before loading onto the Affymetrix GenFlex probe array cartridge. The probe array was then incubated for 2 h at 45° C. at constant rotation (60 rpm). The remaining Adaptor mix was removed from the GenFlex cartridge, and replaced with the template in a hybridization buffer (100 mM MES, 1 M NaCl, 20 mM EDTA, 0.01% Tween 20, 1× Denhardt's). The template hybridisation mix was heated to 95° C. for 5 min and subsequently cooled and maintained at 40° C. for 5 min before loading onto the Affymetrix GenFlex probe array cartridge and hybridised for 2 h at 45° C. at constant rotation (60 rpm). The washing and staining procedure was performed in the Affymetrix Fluidics Station. The probe array was exposed to 2 washes in 6×SSPE-T at 25° C. followed by 12 washes in 0.5×SSPE-T at 40° C. The biotinylated Template oligonucleotide was stained with a streptavidin-phycoerythrin conjugate, final concentration 2 μg/μl (Molecular Probes, Eugene, Oreg.) in 6×SSPE-T for 10 min at 25° C. followed by 6 washes in 6×SSPE-T at 25° C.

The probe arrays were scanned at 560 nm using a confocal laser-scanning microscope with an argon ion laser as the excitation source (Hewlett Packard GeneArray Scanner G2500A). The readings from the quantitative scanning were analysed by the Affymetrix Gene Expression Analysis Software. The results are depicted in Scheme 1.

[image]

The Array analysis shows that the framing regions are able to direct the position of the codon even in the case that all three codons are identical. The designed probes will only detect codons with the correct framing region allowing distinguishing as to which position the codon is positioned. Only one deletion in both framing regions reduces significantly the hybridization of the template. Thus, the framing sequence may be used to obtain information about the position of a specific codon and the point in the reaction history when a given reaction of a chemical entity has occurred.

### Example 2

Selection and Array Detection of DNA Template Coding for Dinitrophenyl (DNP).

This example shows the possibility to decode templates using microarray after a library have been subjected to selection.

For analysis of DNP (dinitrophenyl) coupled to a DNA template the Affymetrix Genflex array was used. Ten array oligonucleotides were randomly selected and ten adaptor oligonucleotides were designed with a sequence complementary to different probes on the microarray (se below). The adaptor oligonucleotides also harboured a portion complementary to the template codons. Five templates were constructed whereof one of the templates encodes the small molecule DNP. All the templates contain three codons (see below) where the middle codon is indicated as underlined. In this example, the codons are directly adjacent to each other but could also be separated by a framing sequence for site detections as shown above. The final setup on the array is shown below. For selection of the oligo encoding DNP, each of the five templates were mixed in 100 μl 10 mM Tris-HCl pH 7.9 to a final concentration of 2 μM. 5 μl of rabbit anti-dinitrophenyl antibody (DAKO) was added and incubated for 2 h at 25° C. The mixture was then incubated with 50 ul Protein A sepharose (Amersham Biosciences) for 2 h at 25° C. After incubation the Protein A sepahrose beads were precipitated and washed three times with 50 mM Tris-HCl pH7.9, 0.5M NaCl, 5 mM MgCl₂, 0.05% SDS and then two times with the same buffer without SDS. The binding substances were eluted with 100 mM Glycin-HCl pH 2.8 and immediately brought to neutral pH by adding 5 μl 2M Tris-HCl pH 7.9.

GenFlex hybridisation and scanning. Prior to hybridization, the Adaptor mix (100 pM each in final concentration) in a hybridization buffer (100 mM MES, 1 M NaCl, 20 mM EDTA, 0.01% Tween 20, 1× Denhardt's), was heated to 95° C. for 5 min and subsequently cooled to 40° C. and maintained at this temperature for 5 min before loading onto the Affymetrix GenFlex probe array cartridge. The probe array was then incubated for 2 h at 45° C. at constant rotation (60 rpm). The Adaptor mix was removed from the GenFlex cartridge, and replaced with 5 μl of the eluted template in the hybridization buffer (100 mM MES, 1 M NaCl, 20 mM EDTA, 0.01% Tween 20, 1× Denhardt's). The template hybridisation mix was heated to 95° C. for 5 min and subsequently cooled to 40° C. at maintained at that temperature for 5 min before loading onto the Affymetrix GenFlex probe array cartridge and hybridised for 2 h at 45° C. at constant rotation (60 rpm). The washing and staining procedure was performed in the Affymetrix Fluidics Station. The probe array was exposed to 2 wash in 6×SSPE-T at 25° C. followed by 12 washes in 0.5×SSPE-T at 40° C.

The biotinylated template oligonucleotide was stained with a streptavidin-phycoerythrin conjugate, final concentration 2 μg/μl (Molecular Probes, Eugene, Oreg.) in 6×SSPE-T for 10 min at 25° C. followed by 6 washes in 6×SSPE-T at 25° C. The probe arrays were scanned at 560 nm using a confocal laser-scanning microscope with an argon ion laser as the excitation source (Hewlett Packard GeneArray Scanner G2500A). The readings from the quantitative scanning were analysed by the Affymetrix Gene Expression Analysis Software.

Array Oligos:

5′GAGTAGCCTTCCCGAGCATT3′; 5′AAACCATCGACTCACGGGAT-
3′;
5′ATTGACCAAACTGCGGTGCG3′; 5′ATTAACTCGACTGCCGCGTG-
3′;
5′AACAACGATGAGACCGGGCT3′; 5′TGCCCTATTGTTGCGTCGGA-
3′;
5′TCTTCTAGTTGTCGAGCAGG3′; 5′TAATCTAATTCTGGTCGCGG-
3′;
5′TGTGATAATTTCGACGAGGC3′; 5′GTGATTAAGTCTGCTTCGGC-
3′

Adaptor Oligos:

| | |
|---|---|
| 1. | 5′-TTTGGGTTTGCCCCTTTTCCAATGCTCGGGAAGGCTACCT-3′ |
| 2. | 5′-TTGGTTGGTTGGTTGGTTGGATCCCGTGAGTCGATGGTTT-3′ |
| 3. | 5′-TGGGTTTGGGGTTTGGGTTTCGCACCGCAGTTTGGTCAAT-3′ |
| 4. | 5′-GTGTGTGTGTTGTGTGTGTGCACGCGGCAGTCGAGTTAAT-3′ |
| 5. | 5′-GTGTTGTTGTTGTGGTGGTGAGCCCGGTCTCATCGTTGTT-3′ |
| 6. | 5′-GGTTGGTTGGTTTGGGTTTGTCCGACGCAACAATAGGGCA-3′ |
| 7. | 5′-GTTTGGGTTTTTGGTTGGTTCCTGCTCGACGGCTAGAAGA-3′ |
| 8. | 5′-TGTGTGTGTGTGGGTTTGGGCCGCGACCAGAATTAGATTA-3′ |
| 9. | 5′-TGTGGTGGTGGTGTGTGTGTGCCRCGTCGAAATTATCACA-3′ |
| 10. | 5′-TTTTTGGGGGGTGTTGTTGTGCCGAAGCAGACTTAATCAC-3′ |

Template Oligos

```
                              1 2 3
Biotin-5′GGAAAAGGGGCAAACCCAAACCAACCAACC-3′
                              4 5 6
Biotin-5′CCAACCAACCAACCAACCAAAAACCCAAAC-3′
                              7 8 9
Biotin-5′AAACCCAAACCCCAAACCCACACACACACA-DNP-3′
                              10 11 12
Biotin-5′CACACACACAACACACACACCACCACCACA-3′
                              13 14 15
Biotin-5′CACCACCACAACAACAACACCCCCCAAAAA-3′
```

[image]

[image]

The result of the experiment shows that probes complementary to codon pair 7/8 and 8/9 can be distinguished from the rest of the codons initially present. Thus, it is possible to identify the codons of a template after a selection has been performed, thereby identifying/decoding the selected compound. The overlapping codon (number 8) found with the probe sets 7/8 and 8/9 will link codon 7 and 9 together and give the final sequence (7-8-9) of codons in the selected template. This experiment is not limited as any size of the library can be used with the same principle.

### Example 3

Selection, Amplification and Detection of DNA Template Encoding DNP.

For selection and amplification of DNP encoded by a DNA template a library of 16×10⁶ was constructed with one of the templates encoding DNP. For array analysis the pre-made Affymetrix genflex array was used. Eighteen array probes were randomly selected and eighteen adaptor oligos were designed. The adaptor Oligos harbours a portion complementary to the Array probes and a portion complementary to a template codon. Each codon comprises 16 nt and the codons were numbered as indicated below. Only three of the oligos on the array should give rise to a signal after array. The other array oligos are negative controls. The library was adjusted to a final volumen of 100 μl in 10 mM Tris-HCl pH 7.9 to a final concentration of 2 μM. 5 μl of rabbit anti-dinitrophenyl antibody (DAKO) was added and incubated for 2 h at 25° C.

The mixture was then incubated with 50 ul Protein A sepharose (Amersham Biosciences) for 2 h at 25° C. After incubation the Protein A sepahrose beads were precipitated and washed three times with 50 mM Tris-HCl pH7.9, 0.5M NaCl, 5 mM MgCl₂, 0.05% SDS and the two times with the same buffer without SDS. The binding substances were eluted with 100 mM Glycin-HCl pH 2.8 and immediately brought to neutral pH by adding 5 μl 2M Tris-HCl pH 7.9. After the selection the sample was subjected to PCR prior to array analysis. The PCR reaction was stopped at 30 cycles and additional polymerase was added before the last 20 cycles. The two library primer sites are underlined. The Library forward primer was synthesized with the two first Guanosine nucleotides inverted (3′-5′ direction) in order to protect the strand from T7 exonuclease digestion. The PCR reaction was performed with the following program: 95° C. for 2 min, and 25 cycles of 95° C. for 30 sec, 52° C. for 30 sec and 72° C. for 30 sec. After the selection the elution of the template was detected with the chromogeniec substrate TMB plus giving rise to a blue colour. No colour reaction could be detected in the control. After the PCR half of the reaction was digested with T7 exonuclease for 30 min at 25° C. The reaction was finally purified on a Bio-Spin P6 column (Bio-Rad).

GenFlex hybridisation and scanning. Prior to hybridization, the Adaptor mix (100 pM each in final concentration) in a hybridization buffer (100 mM MES, 1 M NaCl, 20 mM EDTA, 0.01% Tween 20, 1× Denhardt's), was heated to 95° C. for 5 min and subsequently cooled to 40° C. for 5 min before loading onto the Affymetrix GenFlex probe array cartridge. The probe array was then incubated for 2 h at 45° C. at constant rotation (60 rpm). The Adaptor mix was removed from the GenFlex cartridge, and replaced with 5 μl of the eluted template in the hybridization buffer (100 mM MES, 1 M NaCl, 20 mM EDTA, 0.01% Tween 20, 1× Denhardt's). The Template hybridisation mix was heated to 95° C. for 5 min and subsequently cooled to 40° C. for 5 min before loading onto the Affymetrix GenFlex probe array cartridge and hybridised for 2 h at 45° C. at constant rotation (60 rpm). The washing and staining procedure was performed in the Affymetrix Fluidics Station. The probe array was exposed to 2 wash in 6×SSPE-T at 25° C. followed by 12 washes in 0.5×SSPE-T at 40° C. The biotinylated Template oligo was stained with a streptavidin-phycoerythrin conjugate, final concentration 2 μg/μl (Molecular Probes, Eugene, Oreg.) in 6×SSPE-T for 10 min at 25° C. followed by 6 washes in 6×SSPE-T at 25° C. The probe arrays were scanned at 560 nm using a confocal laser-scanning microscope with an argon ion laser as the excitation source (Hewlett Packard GeneArray Scanner G2500A). The readings from the quantitative scanning were analysed by the Affymetrix Gene Expression Analysis Software

Library Oligonucleotides, in which N indicate any of the nucleotides A, G, C, T.

5′-
GGTAGCCCTCACTCGGCGCCAAGNNNNACTGGCGAGNNNNCTTCGCAAGNNNNAGCGGCTTGGCTAGCCCCGACCG-
3′
5′-
GGTAGCCCTCACTCGGCGCCAAGCCCGACTGGCGAGCGCGCTTCGCAAGGGGGAGCGGCTTGGCTAGCCCCGACCG-
DNP-3′

Array Oligos

3′-TTTGGTAGCTGAGTGCCCTA-5′;
3′-TAACTGGTTTGACGCCACGC-5′
3′-TAATTGAGCTGACGGCGCAC-5′;
3′-TTGTTGCTACTCTGGCCCGA-5′
3′-ACGGGATAACAACGCAGCCT-5′;
3′-AGAAGATCAACAGCTCGTCC-5′
3′-ATTAGATTAAGACCAGCGCC-5′;
3′-ACACTATTAAAGCTGCTCCG-5′
3′-CACTAATTCAGACGAAGCCG-5′;
3′-CAGCTCCTAAGACTTGGACA-5′
3′-GATTGCTTAGACCCTGCACG-5′;
3′-CCTATGATAAGGCACGCACA-5′
3′-CGCTGTGCAAGGCTCGTATA-5′;
3′-CATGATGTAAGCACGCTACC-5′
3′-CAGGAGCGAAGCAGATACTC-5′;
3′-CAGAGCAGAAGCACACACGT-5′
3′-TAAACTGCTTGCATACGGCG-5′;
3′-TATAAGCCTTGCAGCGGACC-5′

Adaptor Oligos

| | |
|---|---|
| 1. | 5′-GCCAGTCGTGCTTGGCATCCCGTGAGTCGATGGTTT-3′ |
| 2. | 5′-GCCAGTCGTCCTTGGCCGCACGGCAGTTTGGTCAAT-3′ |
| 3. | 5′-GCCAGTCGATCTTGGCCACGCGGCAGTCGAGTTAAT-3′ |
| 4. | 5′-GCCAGTCTAACTTGGCAGCCCGGTCTCATCGTTGTT-3′ |
| 5. | 5′-GCCAGTGTAGCTTGGCTCCGACGCAACAATAGGGCA-3′ |
| 6. | 5′-GCGAAGGACCCTCGCCCCTGCTCGACAACTAGAAGA-3′ |
| 7. | 5′-GCGAAGGTAACTCGCCCCGCGACCAGAATTAGATTA-3′ |
| 8. | 5′-GCGAAGGCATCTCGCCGCCTCGTCGAAATTATCACA-3′ |
| 9. | 5′-GCGAAGGGGTCTCGCCGCCGAAGCAGACTTAATCAC-3′ |
| 10. | 5′-CGCAAGGTGTCTCGCCACAGGTTCAGAATCCTCGAC-3′ |
| 11. | 5′-GCCGCTGTTCCTTGCGGCACGTCCCAGATTCGTTAG-3′ |
| 12. | 5′-GCCGCTGCAACTTGCGACACGCACGGAATAGTATCC-3′ |
| 13. | 5′-GCCGCTCTAGCTTGCGATATGCTCGGAACGTGTCGC-3′ |
| 14. | 5′-GCCGCTGGGTCTTGCGCCATCGCACGAATGTAGTAC-3′ |
| 15. | 5′-GCCGCTGACCCTTGCGCTCATAGACGAAGCGAGGAC-3′ |
| 16. | 5′-GCCAGTCGGGCTTGGCTGCACACACGAAGACGAGAC-3′ |
| 17. | 5′-GCGAAGCGCGCTCGCCGCGGCATACGTTCGTCAAAT-3′ |
| 18. | 5′-GCCGCTCCCCCATGCGCCAGGCGACGTTCCGAATAT-3′ |

Primers

| | |
|---|---|
| Forward: | 5′ Biotin-GᵢₙGᵢₙTAGCCCTCACTCGGC-3′ |
| Reverse: | 5′-CGGTCGGGGCTAGCCAA-3′ |

In this example it has have shown that it is possible to capture a small molecule attached to a DNA template in a library consisting of 16 million different templates. After the PCR the sample was subjected to Array analysis.

1. A method for obtaining structural information about an encoded molecule, wherein the encoded molecule has been produced by a process comprising reacting a plurality of chemical entities, said chemical entities being coded for by codons on a nucleic acid template, the method comprising the steps of
i) providing an array comprising a plurality of single stranded nucleic acid probes immobilized in discrete areas of a solid support, wherein the nucleic acid probes are capable of hybridising to a codon of the template,
ii) adding the nucleic acid template or a sequence complementary thereto, to the array under conditions which allow for hybridisation,
iii) observing the discrete areas of the support in which an hybridisation event has occurred.

2. The method according to claim 1, wherein the chemical entities are precursors for a structural unit appearing in the encoded molecule.

3. The method according to claim 1 or 2, wherein the process for producing the encoded molecule comprises transferring the chemical entities to a nascent encoded molecule by a building block, which further comprises an anti-codon.

4. The method according to claim 3, wherein the information of the anti-codon is transferred in conjunction with the chemical entity to the nascent encoded molecule.

5. The method according to any of the claims 1 to 4, wherein the chemical entities are reacted without enzymatic interaction to produce the encoded molecule.

6. The method according to any of the claims 1 to 5, wherein the template comprises two or more codons.

7. The method according to any of the claims 1 to 6, wherein the template comprise three or more codons.

8. The method according to any of the claims 1 to 7, wherein the nucleic acid probe of the array is hybridised to a template through an adapter oligonucleotide having a sequence complementing the probe as well as one or more codons of the template.

9. The method according to any of the preceding claims, wherein neighbouring codons of the template are spaced be a framing sequence.

10. The method according to claim 9, wherein the framing sequence positions the reaction of a chemical entity in the synthesis history of the encoded molecule.

11. The method according to any of the claims 1 to 10, wherein a probe of the array is capable of hybridising to two codons of the template or a sequence complementary to the sequence.

12. The method according to any of the preceding claims, wherein a nucleic acid probe of the array is capable of hybridising to all codons of a template.

13. The method according to any of the claims 1 to 10, wherein a nucleic acid probe is capable of hybridising to all but one codon of the template, or less.

14. The method according to any of the claims 1 to 13, wherein the encoded molecule is attached to the template in step ii).

15. The method according to any of the claims 1 to 13, wherein the template is detached from the encoded molecule.

16. The method according to any of the preceding claims, wherein the template or a sequence complementary thereto prior to the addition to the array has been part of a library of complexes each comprising an encoded molecule attached to the template which encodes said molecule.

17. The method according to claim 16, wherein the library has been subjected to a condition which have partitioned a complex having a predetermined property from the remained of the library and the template of said complex has been amplified.

18. The method according to any of the preceding claims, wherein a plurality templates or sequences complementary thereto coding for the encoded molecules is added in step ii) to obtain structural information of each of the encoded molecules.

19. The method according to any of the preceding claims, wherein the existence of a hybridisation event is measured through labelling of the template.

20. The method according to any of the claims 1 to 19, wherein the hybridisation event is measured by the emission of light in a scanner.

21. The method according to claim 19 and 20, wherein the relative intensity of light in each discrete spot is measured.
